(19) United States
(12) Patent Application Publication
(10) Pub. No.: US 2005/0222516 A1
(43) Pub. Date: Oct. 6, 2005

(54) Body fat measurement apparatus

(75) Inventors: Yasuhiro Kasahara, Tokyo (JP); Yuka Honda, Tokyo (JP); Kiharu Sunako, Tokyo (JP)

Correspondence Address: MCDERMOTT WILL & EMERY LLP, 600 13TH STREET, N.W., WASHINGTON, DC, 20005-3096, US

(73) Assignee: TANITA CORPORATION

(21) Appl. No.: 11/097,133

(22) Filed: Apr. 4, 2005

(30) Foreign Application Priority Data
Apr. 5, 2004 (JP) 2004-111349

(51) Int. Cl. A61B 5/05

(52) U.S. Cl. 600/547

(57) ABSTRACT

Disclosed is a body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated. According to the present invention, said apparatus further comprising: an electrode support unit; a lateral width measurement unit; and an arithmetic unit, wherein said electrode support unit supports the impedance measurement electrode system so that the electrodes thereof are contact with front surface of the abdomen of the person under test for measuring impedance of the abdomen, said lateral width measurement unit measures lateral width of the abdomen of the person under test, and said arithmetic unit calculates the index of body fat of the person under test on the basis of the impedance of the abdomen and the lateral width.

### BACKGROUND OF THE INVENTION

1. Field of the Invention

The present invention relates to a body fat measurement apparatus for measuring abdomen's impedance of a person under test and for calculating an index of body fat.

2. Prior Art

In the past, there has been developed such body fat measuring apparatus that comprises impedance measurement electrodes adapted for contact with hands and feet of a person under test to measure impedance thereof, on the basis of which an index of body fat is estimated.

Another body fat measurement apparatus for providing body fat data according to tomogram picture resulted from “X-ray CT”, “Impedance CT” and other CT methods or “MRI” method has also been developed in order to precisely derive, in particular, visceral fat area and subcutaneous fat area for the index of body fat. Other apparatus using “DXA” method has additionally been known in order to precisely derive, in particular, visceral fat rate and subcutaneous fat rate for the index of body fat.

Furthermore, various researches and techniques have been disclosed in which impedance measurement electrodes are directly contact with the surface of abdomen of a person under test to measure impedance thereof, on the basis of which an index of body fat is calculated. For example, a research has been done in which visceral fat mass or visceral fat area is calculated in correlation with “CT” methods on the basis of waist size of a person under test and impedance value measured using a pair of current supplying electrodes positioned on opposite surfaces, i.e. front and back sides of abdomen and a pair of voltage measurement electrodes positioned on both side surfaces of abdomen. (Refer to Non-Patent Document 1.)

Another research has been done in which a belt is wrapped around abdomen of a person under test in such manner that impedance measurement electrodes are contact with front surface of abdomen to measure abdomen's impedance, on the basis of which subcutaneous fat is estimated. (Refer to Non-Patent Document 2.)

An additional research has been done in which impedance of around abdomen is measured while changing part of body to be measured and frequency of electric current supplied thereto, and the part of body and the frequency suitable for estimation for visceral fat area is determined. (Refer to Non-Patent Document 3.)

Furthermore, a body fat measurement apparatus has been disclosed in which visceral fat mass is calculated on the basis of voltage measured using electrodes positioned on outer peripheral surface of a person under test at such interval that is sufficiently shorter relative to outer peripheral length of body of the person and of characteristic amount reflecting the dimension of human body represented by e.g. whole cross section area, outer peripheral length of cross section area, or vertical and horizontal width of cross section. (Refer to e.g. Patent Document 1.)

In addition, an impedance measurement apparatus has been disclosed in which a contact surface means on which electrodes are positioned with fixed interval is contact with a part of body of a person under test to measure impedance thereof. (Refer to e.g. Patent Document 2.)

Non-Patent Document 1: Literature by Miwa Yana, entitled “Development for Measurement of Visceral Fat Using Abdominal Bioimpedance Method”;

Non-Patent Document 2: Literature by Hermann Scharfetter and five others, entitled “Assessing abdominal fatness with local bioimpedance analysis: Basics and experimental findings”;

Non-Patent Document 3: Literature by Hideaki Komiya and one other, entitled “Research for Estimation of Visceral Fat Area Using Multi-Frequency Impedance Method”, Fatness Research, 2003, Vol. 9, No. 1;

Patent Document 1: Japanese Patent No. 3396674; and

Patent Document 2: patent application No. 2000-128049.

However, the prior art apparatus designed to estimate impedance of truck portion of a person under test, based on the impedance measured using the electrodes adapted for contact with his/her hands or feet, as described above, is defective in that reliability of estimation for impedance of trunk portion becomes extremely lower if the impedance may erroneously be measured or can't be measured at all due to the fact that any of limbs of the person with which the electrodes are contact has some trouble, for example.

If “CT” or “DXA” method is used to derive the index of body fat then “CT”, method is only effective to measure body fat area for the index of body fat, but “DXA” method can't measure the body fat area. Accordingly, in order to derive the index of body fat including the body fat area, it is necessary to calculate it using at least both “CT and “DXA” methods. However, the apparatus using said methods are commonly designed to be operated in the medical field by those persons having expert knowledge and techniques, and in addition, the apparatus itself is very expensive. Therefore, it was not common to easily derive the index of body fat.

Furthermore, in the apparatus wherein the impedance measurement electrodes are positioned at opposite sides of the abdomen, or the belt is wrapped around the peripheral of abdomen, or the characteristic amount reflecting the dimension of human body is represented by total cross section area, outer peripheral length of cross section area, or vertical and horizontal width of cross section, it is very difficult for a person under test to install the apparatus by himself/herself so that the electrodes or other portions of the apparatus are positioned at the backside of the person, as described above, especially in case where the person under test is confined or almost confined to one's bed for a long time. Such installation of the apparatus to the person under test takes much labor even with the assistance by an attendant. In particular, if it is necessary to separately measure the dimension of the part of body, additional time and labor becomes necessary, which makes the measurement substantially impractical.

Moreover, in the apparatus wherein the electrodes positioned on the contact surface means are contact with a part of body of a person under test to measure the impedance thereof, it tends to produce any difference in measurement region defined by electrode contact position, depending on the dimension of abdomen of the person, which may cause variation in measurement condition, due to the fact that there is significant personal difference in distribution of biological tissue such as subcutaneous fat, visceral fat, and muscle, if measurement is made on abdomen, not on limbs of the person under test.

In view of the above it is an object of the present invention to solve the above-mentioned problems in the prior art and to provide an improved body fat measurement apparatus that allows ease, but precise measurement of impedance at front surface of abdomen of a person under test and that allows reliable calculation of an index of body fat based on impedance of abdomen and lateral width of abdomen.

### SUMMARY OF THE INVENTION

In order to attain such object the present invention provides, in a first aspect, a body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated, said apparatus further comprising: an electrode support unit; a lateral width measurement unit; and an arithmetic unit, wherein
said electrode support unit supports the impedance measurement electrode system so that the electrodes thereof are contact with front surface of the abdomen of the person under test for measuring impedance of the abdomen,
said lateral width measurement unit measures lateral width of the abdomen of the person under test, and
said arithmetic unit calculates the index of body fat of the person under test on the basis of the impedance of the abdomen and the lateral width.

According to one embodiment of the present invention said electrode support unit may be formed from flexible member.

The present invention provides, in a second aspect, a body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated, said apparatus further comprising: an electrode support unit; an electrode position shift unit; and an arithmetic unit, wherein
said electrode support unit supports the impedance measurement electrode system so that the electrodes thereof are contact with front surface of the abdomen of the person under test for measuring impedance of the abdomen,
said electrode position shift unit moves the electrode support unit according to lateral width of the abdomen so that the electrodes of the impedance measurement electrode system are set at suitable position on the front surface of the abdomen, and
said arithmetic unit calculates the index of body fat of the person under test on the basis of the impedance of the abdomen.

According to one embodiment of the present invention said electrode support unit may support the impedance measurement electrode system so that the pair of voltage measurement electrodes is positioned between the pair of current supplying electrodes.

According to another embodiment of the present invention said electrode support unit may include an electrode direction change unit, said electrode direction change unit allows at least one of the pair of current supplying electrodes and the pair of voltage measurement electrodes to change in contact direction in order to suit to the shape of the abdomen.

According to further embodiment of the present invention the body fat measurement apparatus may further comprise a frequency switching unit, said frequency switching unit switches frequency of AC current supplied to the pair of current supplying electrodes in order to conduct impedance measurement at two or more frequencies of at least one of higher and lower frequency ranges.

According to yet further embodiment of the present invention said index of body fat may include at least one of the followings: total fat area of abdomen, subcutaneous fat area, subcutaneous fat thickness, visceral fat area, fat rate of trunk portion, fat rate of whole body, and ratio of subcutaneous fat to visceral fat.

### EFFECTS OF THE INVENTION

The present invention is directed toward a body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated. According to the present invention, said apparatus further comprises: an electrode support unit; a lateral width measurement unit; and an arithmetic unit, wherein said electrode support unit supports the impedance measurement electrode system so that the electrodes thereof are contact with front surface of the abdomen of the person under test for measuring impedance of the abdomen, said lateral width measurement unit measures lateral width of the abdomen of the person under test, and said arithmetic unit calculates the index of body fat of the person under test on the basis of the impedance of the abdomen and the lateral width. Accordingly, there is no need for the impedance measurement electrodes or some waist measuring instrument positioned at opposite sides of the body of the person under test so that the body is sandwiched therebetween. Instead, simply pressing the apparatus against the front surface of abdomen of the person under test can easily put the apparatus into proper measurement position. As the result, not only a normal person but also a handicapped person having some trouble in any of limbs can easily be given the index of body fat, even with confined in the bed.

The electrode support unit may be formed from some flexible member. Accordingly, the electrodes of the impedance measurement electrode system can closely be contact to the abdomen of the person under test, which can reduce any error caused by poor contact condition of the electrodes.

Furthermore, the present invention is directed toward a body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated. According to the present invention, said apparatus further comprises: an electrode support unit; an electrode position shift unit; and an arithmetic unit, wherein said electrode support unit supports the impedance measurement electrode system so that the electrodes thereof are contact with front surface of the abdomen of the person under test for measuring impedance of the abdomen, said electrode position shift unit moves the electrode support unit according to lateral width of the abdomen so that the electrodes of the impedance measurement electrode system are set at suitable position on the front surface of the abdomen, and said arithmetic unit calculates the index of body fat of the person under test on the basis of the impedance of the abdomen. Accordingly, by physically moving the electrode position, an optimized measurement condition suitable for abdomen of the person under test can easily be attained, irrespective of the dimension of the abdomen.

The electrode support unit may support the impedance measurement electrode system so that the pair of voltage measurement electrodes is positioned between the pair of current supplying electrodes. Accordingly, stable impedance measurement can be attained that is comparable with that of the prior art bioimpedance measurement apparatus for measuring one part of body, as described earlier.

The electrode support unit may include an electrode direction change unit, said electrode direction change unit allows at least one of the pair of current supplying electrodes and the pair of voltage measurement electrodes to change in contact direction in order to suit to the shape of the abdomen of the person under test. Accordingly, the electrodes of the impedance measurement electrode system can closely be contact to the abdomen of the person under test, which can mitigate any error caused by poor contact condition of the electrodes.

The body fat measurement apparatus may further comprise a frequency switching unit, said frequency switching unit switches frequency of AC current supplied to the pair of current supplying electrodes in order to conduct impedance measurement at two or more frequencies of at least one of higher and lower frequency ranges. Accordingly, it is possible to provide the impedance contributing to a portion of biological tissue such as fat tissue, muscle tissue, etc. at each of frequencies. As the result, measurement at multi-frequency can provide the index of body fat with higher precision.

The index of body fat may include at least one of the followings: total fat area of abdomen, subcutaneous fat area, subcutaneous fat thickness, visceral fat area, fat rate of trunk portion, fat rate of whole body, and ratio of subcutaneous fat to visceral fat. Therefore, the index of body fat that can previously be measured only by any highly expensive apparatus using “CT” or “DXA” method can, now, easily be measured by an inexpensive apparatus according to the present invention.

### BRIEF DESCRIPTION OF THE DRAWINGS

The present invention will be described in more detail with reference to the accompanying drawings, in which:

FIG. 1 is a top view of a body fat measurement apparatus according to a first embodiment of the present invention when it is in use;

FIG. 2 is a cross section view of the body fat measurement apparatus taken along the line A-A in FIG. 1;

FIG. 3 is an electrical block diagram of the body fat measurement apparatus;

FIG. 4 is a model view of front surface of abdomen of a person under test modeled by an electrical equivalent circuit of current path therethrough;

FIG. 5 is a model view of cells illustrating different current paths depending on frequency ranges in electrolyte tissue;

FIG. 6 is a flow chart illustrating operation of the body fat measurement apparatus;

FIG. 7 is a graph for comparing total fat area of abdomen resulted by “CT” method with that calculated by the regression equation;

FIG. 8 is a graph for comparing subcutaneous fat area resulted by “CT” method with that calculated by the regression equation;

FIG. 9 is a graph for comparing visceral fat area resulted by “CT” method with that calculated by the regression equation;

FIG. 10 is a graph for comparing fat rate of trunk portion resulted by “DXA” method with that calculated by the regression equation;

FIG. 11 is a graph for comparing fat rate of whole body with fat rate of trunk portion;

FIG. 12 is a top view of a body fat measurement apparatus according to a second embodiment of the present invention when it is in use;

FIG. 13 is a cross section view of the body fat measurement apparatus taken along the line A-A in FIG. 12;

FIG. 14 is a top view of a body fat measurement apparatus according to a third embodiment of the present invention when it is in use;

FIG. 15 is a cross section view of the body fat measurement apparatus taken along the line A-A in FIG. 14;

FIG. 16 is a top view of a body fat measurement apparatus according to a fourth embodiment of the present invention when it is in use;

FIG. 17 is a cross section view of the body fat measurement apparatus taken along the line A-A in FIG. 16;

FIG. 18 is a top view of a body fat measurement apparatus according to a fifth embodiment of the present invention when it is in use;

FIG. 19 is a cross section view of the body fat measurement apparatus taken along the line A-A in FIG. 18; and

FIG. 20 is a graph illustrating relationship between subcutaneous fat thickness resulted by “CT” method and “Z_{6.25}”.

### BEST MODE FOR EMBODYING THE INVENTION

A body fat measurement apparatus according to the present invention comprises an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated. The apparatus further comprises: an electrode support unit; a lateral width measurement unit; and an arithmetic unit, wherein said electrode support unit supports the impedance measurement electrode system so that the electrodes thereof are contact with front surface of the abdomen of the person under test for measuring impedance of the abdomen, said lateral width measurement unit measures lateral width of the abdomen of the person under test, and said arithmetic unit calculates the index of body fat of the person under test on the basis of the impedance of the abdomen and the lateral width.

The electrode support unit may be formed from some flexible member.

Furthermore, a body fat measurement apparatus according to the present invention comprises an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated. The apparatus further comprises: an electrode support unit; an electrode position shift unit; and an arithmetic unit, wherein said electrode support unit supports the impedance measurement electrode system so that the electrodes thereof are contact with front surface of the abdomen of the person under test for measuring impedance of the abdomen, said electrode position shift unit moves the electrode support unit according to lateral width of the abdomen so that the electrodes of the impedance measurement electrode system are set at suitable position on the front surface of the abdomen, and said arithmetic unit calculates the index of body fat of the person under test on the basis of the impedance of the abdomen.

The electrode support unit may support the impedance measurement electrode system so that the pair of voltage measurement electrodes is positioned between the pair of current supplying electrodes.

The electrode support unit may include an electrode direction change unit, said electrode direction change unit allows at least one of the pair of current supplying electrodes and the pair of voltage measurement electrodes to change in contact direction in order to suit to the shape of the abdomen of the person under test.

The body fat measurement apparatus may further comprise a frequency switching unit, said frequency switching unit switches frequency of AC current supplied to the pair of current supplying electrodes in order to conduct impedance measurement at two or more frequencies of at least one of higher and lower frequency ranges.

The index of body fat may include at least one of the followings: total fat area of abdomen, subcutaneous fat area, subcutaneous fat thickness, visceral fat area, fat rate of trunk portion, fat rate of whole body, and ratio of subcutaneous fat to visceral fat.

### First Embodiment

A first embodiment of the present invention discloses a body fat measurement apparatus comprising an electrode support unit having an impedance measurement electrode system consisting of a pair of current supplying electrodes and a pair of voltage measurement electrodes all disposed on a contact surface of the electrode support unit with fixed interval, and arm portions slidably movable to suit to lateral width of abdomen of a person under test, wherein the electrodes of the impedance measurement electrode system are positioned on front surface of abdomen about the center of abdomen, i.e. a navel position, relative to lateral width of abdomen, so that any effect of dimension of impedance measurement region relative to the lateral width of abdomen on the abdomen's impedance is corrected to allow for a plurality of persons under test to be measured in the same measurement condition. Furthermore, an index of body fat can easily be calculated using a regression equation obtained as the result of correlation of such corrected abdomen's impedance with an index of body fat preliminary derived by “CT” or “DXA” method. Moreover, impedance measurement is conducted by switching between two frequency ranges: higher frequency range and lower frequency range. Accordingly, any impedance strongly reflecting muscle tissue or subcutaneous fat tissue in abdomen can be measured, whereby the index of body fat can be calculated with higher precision.

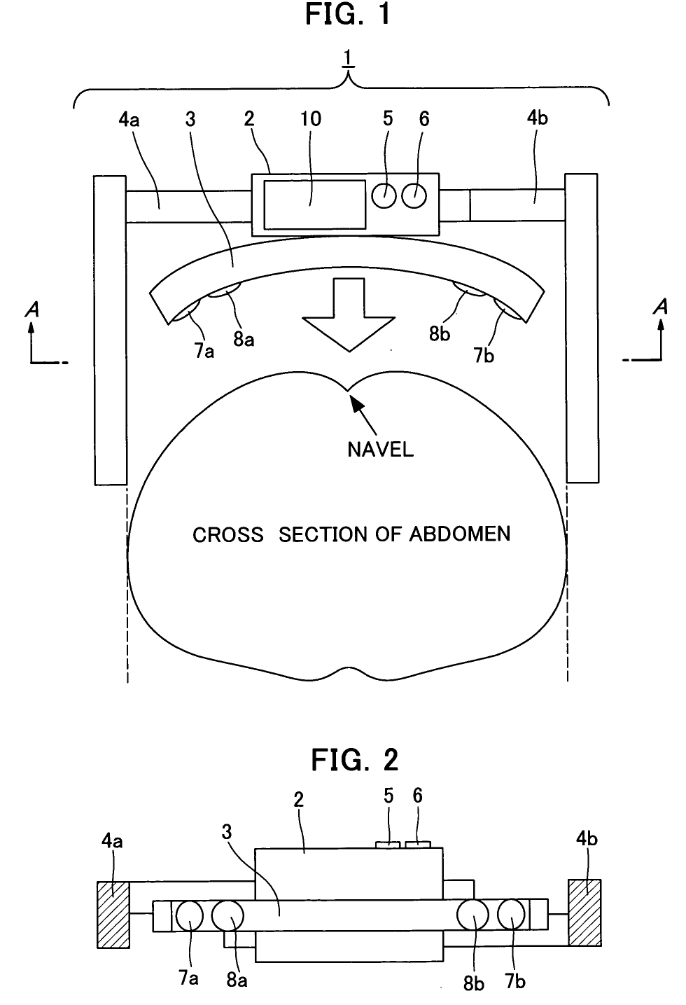

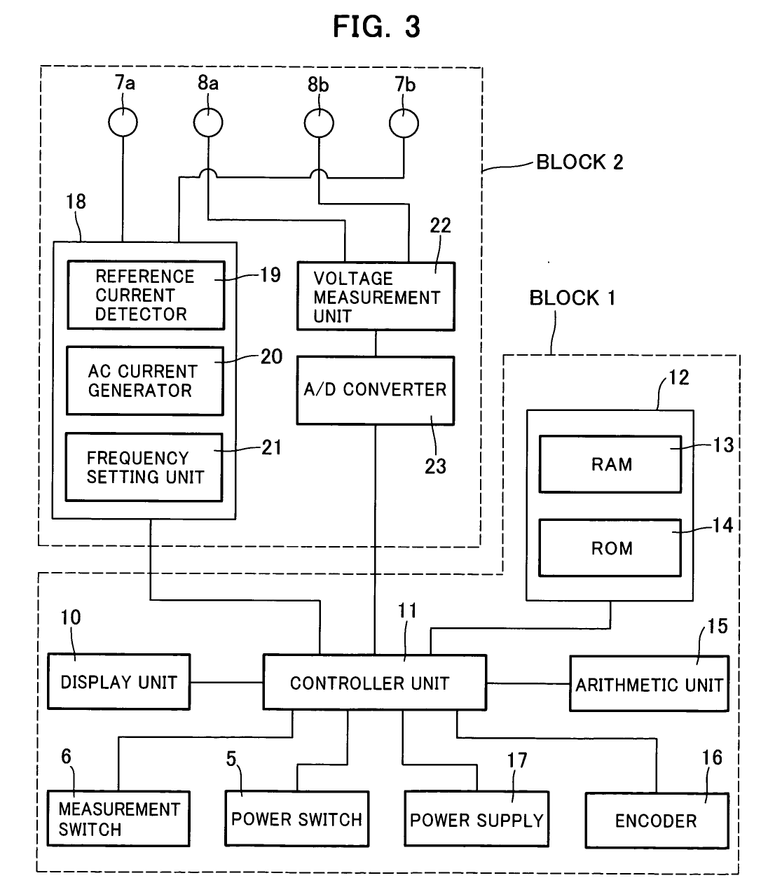

Referring now to FIGS. 1 to 3, a body fat measurement apparatus according to a first embodiment of the present invention will be described in more detail. In particular, FIG. 1 is a top view of a body fat measurement apparatus when it is in use; FIG. 2 is a cross section view of the body fat measurement apparatus taken along the line A-A in FIG. 1; and FIG. 3 is an electrical block diagram of the body fat measurement apparatus.

As shown in FIG. 1, the body fat measurement apparatus 1 comprises a body portion 2, an electrode support unit 3, and left and right arm portions 4a and 4b.

The body portion 2 of the apparatus 1 includes a power switch 5 for turning the apparatus ON or OFF, a measurement switch 6 for starting measurement, and a display unit 10 for displaying guidance message for measurement, result of measurement, etc.

The electrode support unit 3 is designed to be mounted to a side surface of the body portion 2 and is formed from some flexible member to provide a curved leaf spring so that one side thereof that is to be contact with front surface of abdomen of a person under test is directed inwardly, as shown in FIG. 1. An impedance measurement electrode system 9 including a pair of current supplying electrodes 7a and 7b and a pair of voltage measurement electrodes 8a and 8b is provided near both end portion of the electrode support unit 3. In particular, the pair of voltage measurement electrodes 8a and 8b is positioned between the pair of current supplying electrodes 7a and 7b.

Accordingly, when the body fat measurement apparatus 1 is pressed against the front surface of abdomen of the person under test in the direction as indicated by a black arrow in FIG. 1 then the electrode support unit 3 may be flexed to suit to the shape of abdomen of the person under test. As the result, the electrodes of the impedance measurement electrode system 9 can closely be contact to the front surface of abdomen of the person under test.

The left and right arm portions 4a and 4b each has generally “L” shaped construction. One end of “L” is inserted into the body portion 2 and is supported by some sliding mechanism (not shown). The sliding mechanism acts to sidably move the left and right arm portions 4a and 4b in left and right directions respectively by equal distance from the body portion 2 as the centre. Accordingly, simply by making contact the left and right arm portions 4a and 4b with left and right sides of abdomen of the person under test, respectively, the body portion 2 of the apparatus can always be positioned at the center of front surface of abdomen of the person under test. As the result, the electrodes of the impedance measurement electrode system 9 can symmetrically be positioned about the center of abdomen in left-and-right direction.

Referring now to electrical block diagram of FIG. 3, internal configuration of the body fat measurement apparatus 1 will be described. The block diagram generally consists of block 1 and block 2. In particular, the block 1 includes functions of arithmetic operation, data input and output, storage, etc., and the block 2 includes function of impedance measurement.

In the block 1 the body fat measurement apparatus 1 includes a controller unit 11 for executing all the control functions of the apparatus. A storage unit 12 and an arithmetic unit 15 are connected to the controller unit 11. The storage unit 12 includes a RAM 13 for temporally storing some measurement data, arithmetic result, etc., and a ROM 14 for storing control program for the apparatus, calculation equations or judgment program for index of body fat that is set in advance, frequency of AC current supplied in impedance measurement, etc. The arithmetic unit 15 calculates the impedance value and the index of body fat.

Also connected with the controller unit 11 are an encoder unit 16 and a power supply 17. The encoder unit 16 acts to measure the distance between the arm portions 4a and 4b by measuring the moving distance for which the arm portions 4a and 4b have been moved by the sliding mechanism of the body portion 2. That is to say, a side-to-side distance of abdomen represented by the distance between the arm portions 4a and 4b (hereafter referred to as “lateral width of abdomen”) is automatically measured by the encoder unit 16.

Furthermore, the power switch 5, the measurement switch 6 and the display unit 10 are connected to the controller unit 11.

In block 2 the current supplying electrodes 7a and 7b are connected to a current supply unit 18, and the voltage measurement electrodes 8a and 8b are connected to a voltage measurement unit 22 which is then connected to an A/D converter 23. Finally, both are connected to the controller unit 11 in the block 1.

The current supply unit 18 includes a reference current detector 19, an AC current generator 20, and a frequency setting unit 21. The controller unit 11 controls the frequency setting unit 21 to set the frequency at the predetermined one, on the basis of which the AC current generator 20 generates AC current. This AC current is detected in the reference current detector 19 as the current to be supplied to the person under test, and then, it is supplied to the current supplying electrodes 7a and 7b.

An analog voltage signal picked up by the voltage measurement electrodes 8a and 8b is measured in the voltage measurement unit 22, and then, it is converted to a digital signal in the A/D converter 23.

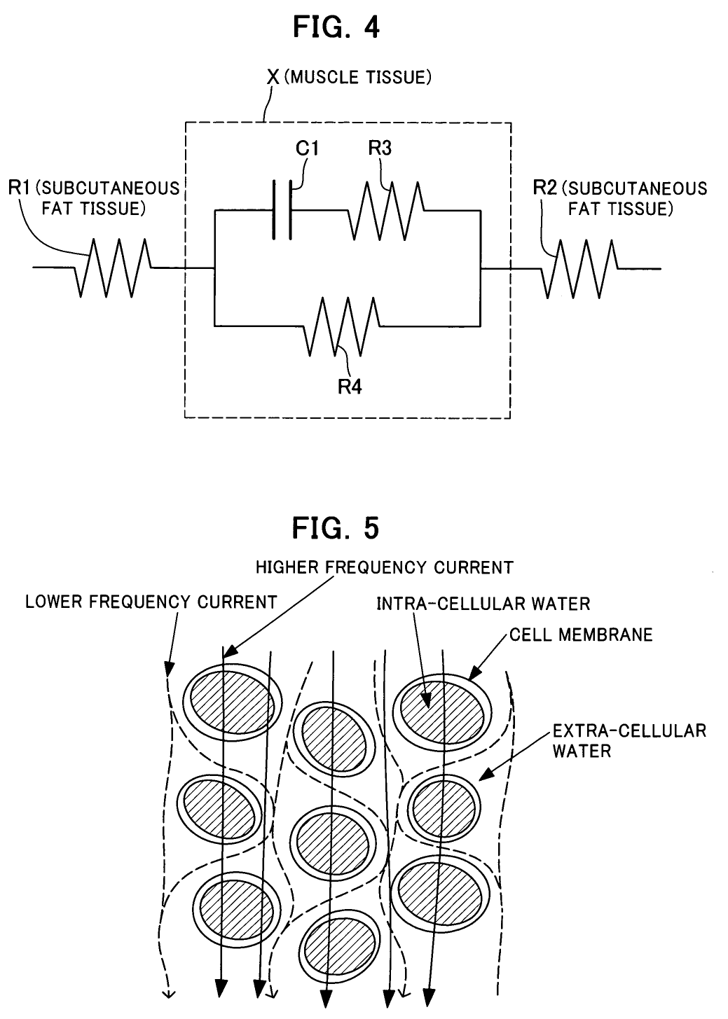

Referring to now to FIGS. 4 and 5, principle of measurement of the abdomen's impedance will be described in more detail. In particular, FIG. 4 is a model view of front surface of abdomen of a person under test modeled by an electrical equivalent circuit of current path therethrough; and FIG. 5 is a model view of cells illustrating different current paths depending on frequency ranges in electrolyte tissue substantially consisting of muscle tissue.

In general, it is considered, in biological tissue, that most of fat free tissue is body water including much electrolyte through which electric current is likely to pass, but fat tissue and bone is non-electrolyte tissue including substantially no electrolyte. Accordingly, at the front surface of abdomen, muscle tissue that is fat free tissue is of electrolyte, but subcutaneous and visceral fat that is fat tissue is of non-electrolyte.

Accordingly, when the current supplying electrodes 7a and 7b are contact with front surface of abdomen of the person under test then electric current is considered to flow through subcutaneous fat tissue into muscle tissue having higher electrical conductively than visceral fat tissue. As shown in FIG. 4, the current path between the current supplying electrodes 7a and 7b is modeled in such manner that subcutaneous fat tissue under the contact positions of electrodes 7a and 7b are represented by resistors “R1” and “R2”, respectively, and muscle tissue is represented by circuit “X”. In particular, the circuit “X” is an electrical equivalent circuit of electrolyte tissue wherein it is formed by a serial connection of capacitor “C1” and resistor “R3” in parallel with a resistor “R4”.

It is noted, here, that the circuit “X” represents electrolyte tissue that is modeled at cell level. As shown in FIG. 5, the electrolyte tissue consists of cells each including intra-cellular water covered by cell membrane, and extra- cellular water outside the cells. The intra-cellular water and extra-cellular water act as resistor, and the cell membrane is considered as insulator, but it has capacity because of very thin in thickness. Accordingly, at lower frequency that is near DC current, the cell membrane becomes to act as insulator so that no current flows through intra-cellular water. However, as the frequency of current is increased it starts to flow through intra-cellular water via cell membrane. As the result, in the electrical equivalent circuit capacitor “C1” means cell membrane, resistor “R3” means intra-cellular water and resistor R4 means extra-cellular water.

In circuit “X”, at lower frequency of current used, the current only flows through resistor R4. On the other hand, at higher frequency of current used, the current flows through the combined resistance of resistors R3 and R4 (i.e. (R3×R4)/(R3+R4)). Because of R4<(R3×R4)/(R3+R4), in the model of front surface of abdomen in FIG. 4, at lower frequency of current supplied, the impedance more strongly affected by subcutaneous fat tissue represented by resistors “R1” and “R2” would be detected, than that affected at higher frequency of current supplied. Inversely, at higher frequency of current supplied, the impedance more strongly affected by muscle tissue would be detected. Accordingly, as the frequency of current is increased the impedance measured becomes more strongly affected by muscle tissue.

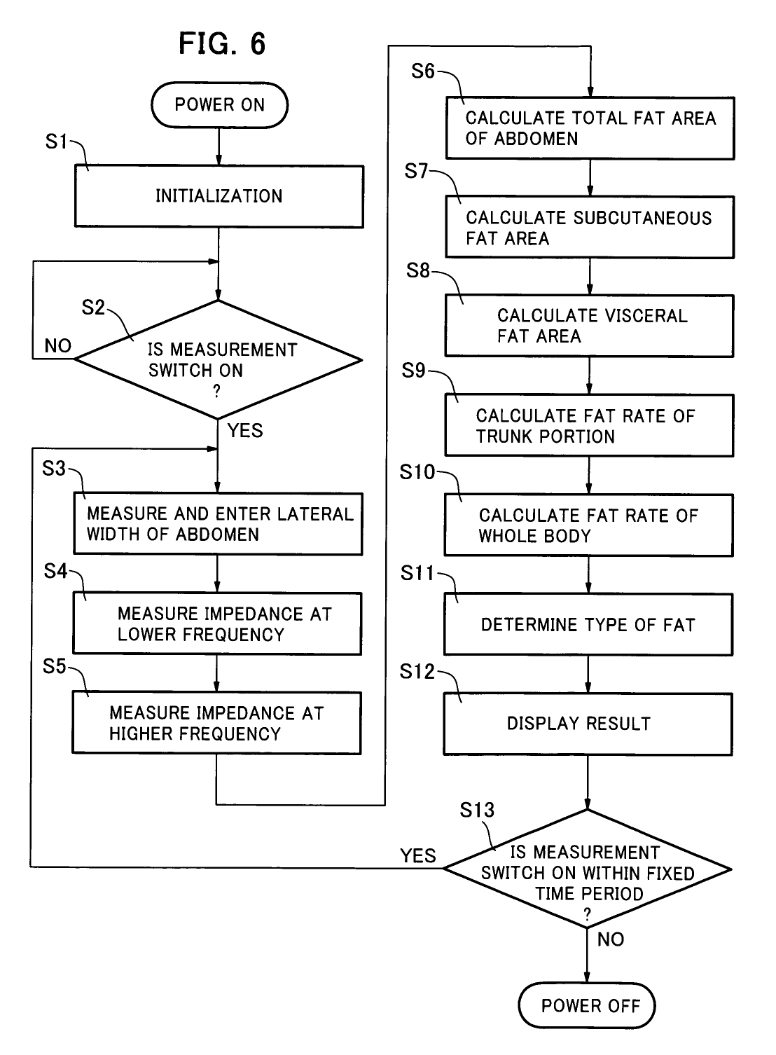

Referring now to FIG. 6 operation of the body fat measurement apparatus will be described in more detail.

First of all, the power switch 5 is pushed to turn ON the body fat measurement apparatus 1. At step Si initial setting of the apparatus is performed and some message is displayed on the display unit 10 informing that the measurement switch 6 is depressed for starting measurement after the apparatus 1 is installed to a person under test. The person under test sidably draws the arm portions 4a and 4b out of the body portion 2 of the apparatus 1 to suit to lateral width of his/her abdomen, and then, lifts the arm portions at the height of his/her navel. Thereafter, the person under test presses the contact surface of the electrode support unit 3 on which the impedance measurement electrode system 9 is positioned against front surface of his/her abdomen to complete installation of the apparatus 1.

At step S2 it is determined whether the measurement switch 6 is depressed or not. If not, the routine proceeds to “NO” branch and the step S2 is repeatedly performed until the measurement switch 6 is depressed, upon which the routine proceeds to “YES” branch. Then, at step S3 the moving distance of the arm portions 4a and 4b drawn out of the body portion 2 is measured by the encoder unit 16 and the arithmetic unit 15 calculates the lateral width of abdomen of the person under test based on the moving distance of the arm portions 4a and 4b.

At step S4 measurement of impedance is conducted at the predetermined lower frequency of current. The impedance value measured is temporally stored in RAM 13. At step S5 measurement of impedance is conducted at the predetermined higher frequency of current. The impedance value measured is temporally stored in RAM 13 in the same manner. It is assumed, here, that lower frequency of current is at 6.25 kHz, higher frequency of current is at 50 kHz, and the impedance values measured at those frequencies are represented by “Z_{6.25}” and “Z₅₀”, respectively.

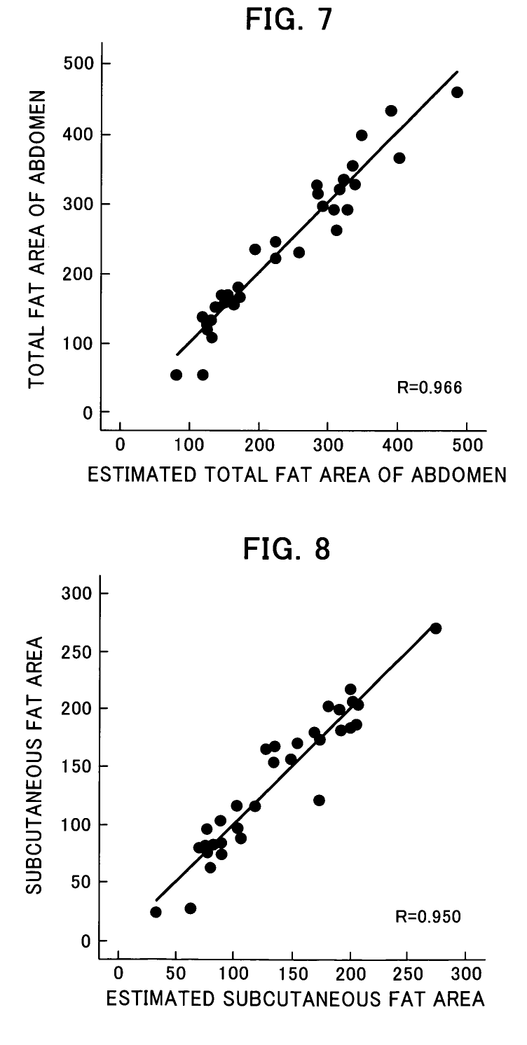

After completion of impedance measurement the routine proceeds to subsequent steps wherein the index of body fat is calculated. In particular, at step S6 total fat area of abdomen is calculated. The controller unit 11 retrieves, from RAM 13, the impedance values measured at lower and higher frequencies, and the lateral width of abdomen of the person under test. In addition, the controller unit 11 retrieves, from ROM 14, a regression equation for calculating total fat area of abdomen, which is obtained as the result of correlation of the total fat area of abdomen preliminary calculated by “CT” method with said impedance values and said lateral width of abdomen. Then, the arithmetic unit 15 calculates the total fat area of abdomen using the regression equation retrieved. The regression equation is written as follows:
Total Fat Area of Abdomen=p₁+q₁×Z_{6.25}+r₁×(Lateral Width of Abdomen)+s₁×Z₅₀/(Lateral Width of Abdomen)
Where p₁, q₁, r₁, and s₁ are constant. In this embodiment, assuming that p₁=−1500, q₁=80, r₁=−77, and s₁=−960, then the total fat area of abdomen calculated by said regression equation is highly correlated with that calculated by “CT” method with a correlation factor of 0.966, as seen in a graph of FIG. 7.

Then, at step S7 the subcutaneous fat area is calculated. In the same manner as the total fat area of abdomen as above, the subcutaneous fat area is calculated using a regression equation which is obtained as the result of correlation of the subcutaneous fat area preliminary calculated by “CT” method with said impedance values and said lateral width of abdomen. The regression equation is written as follows:
Subcutaneous Fat Area=p₂+q₂×Z_{6.25}+r₂×(Lateral Width of Abdomen)+s₂×Z₅₀/(Lateral Width of Abdomen)
Where p₂, q₂, r₂, and s₂ are constant. In this embodiment, assuming that p₂=−350, q₂=−8.0, r₂=35.1, and s₂=500, then the subcutaneous fat area calculated by said regression equation is highly correlated with that calculated by “CT” method with a correlation factor of 0.950, as seen in a graph of FIG. 8.

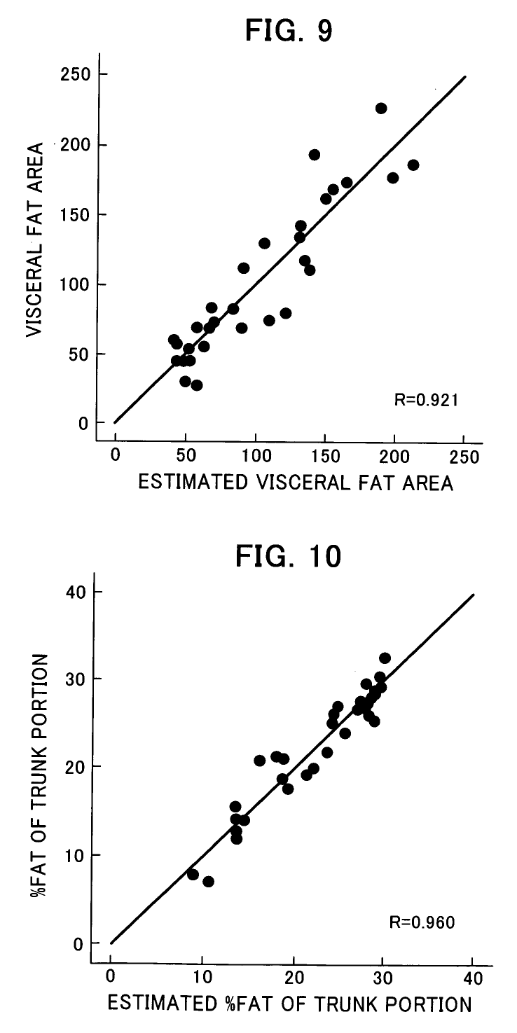

Then, at step S8 the visceral fat area is calculated. In the same manner as the total fat area of abdomen and the subcutaneous fat area as above, the visceral fat area is calculated using a regression equation which is obtained as the result of correlation of the visceral fat area preliminary calculated by “CT” method with said impedance values and said lateral width of abdomen. The regression equation is written as follows:
Visceral Fat Area=p₃+q₃×Z_{6.25}+r₃×(Lateral Width of Abdomen)+s₃×Z₅₀/(Lateral Width of Abdomen)
Where p₃, q₃, r₃, and s₃ are constant. In this embodiment, assuming that p₃=−1030, q₃=90, r₃=−63, and s₃=−1460, then the visceral fat area calculated by said regression equation is highly correlated with that calculated by “CT” method with a correlation factor of 0.921, as seen in a graph of FIG. 9.

Then, at step S9 the fat rate of trunk portion is calculated. The fat rate of trunk portion is calculated using a regression equation which is obtained as the result of correlation of the fat rate of trunk portion preliminary calculated by “DXA” method with said impedance values and said lateral width of abdomen. The regression equation is written as follows:
Fat Rate of Trunk Portion=p₄+q₄×Z_{6.25}+r₄×(Lateral Width of Abdomen)+s₄×(1/Z₅₀/(Lateral Width of Abdomen))
Where p₄, q₄, r₄, and s₄ are constant. In this embodiment, assuming that p₄=90, q₄=−0.99, r₄=−0.825, and s₄=−6980, then the fat rate of trunk portion calculated by said regression equation is highly correlated with that calculated by “DXA” method with a correlation factor of 0.960, as seen in a graph of FIG. 10.

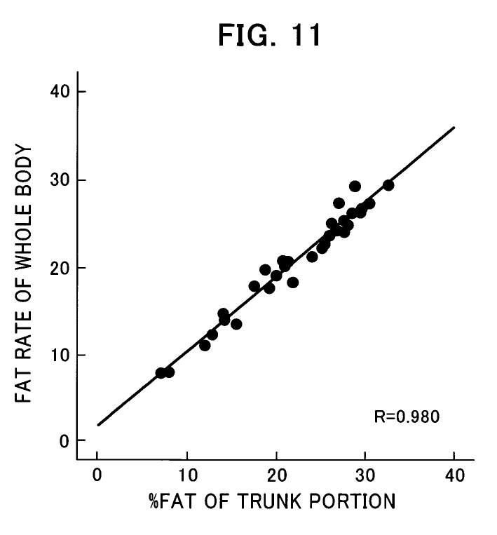

Then, at step S10 the body fat rate of whole body is calculated. In the same manner as the fat rate of trunk portion as above, the body fat rate of whole body is calculated using a regression equation which is obtained as the result of correlation of the body fat rate of whole body preliminary calculated by “DXA” method with said fat rate of trunk portion. The regression equation is written as follows:
Body Fat Rate of Whole body=t×(Fat Rate of Trunk Portion)+u
Where “t” and “u” are constant. In this embodiment, assuming that “t”=0.77 and “u”=3.2 then the fat rate of trunk portion is highly correlated with the body fat rate of whole body calculated by “DXA” method with a correlation factor of 0.980, as seen in a graph of FIG. 11.

Due to the fact that limbs of a person generally have less fat as compared to his/her trunk portion then the body fat rate of whole body can be considered to mainly depend on fat rate of trunk portion. Accordingly, even for a person having some trouble in any of limbs the body fat rate of whole body can be calculated using the regression equation for calculating the body fat rate of whole body from the fat rate of trunk portion, in the same manner as the case of a normal person.

At step S11 type of fat is determined depending on difference between the subcutaneous and visceral fat area calculated in such manner. For example, difference “X” is calculated as follows:
“X”=(Subcutaneous Fat Area)−(sceral Fat Area)
If “X”>0 then type of fat is determined to be subcutaneous fat type, but if “X”≦0 then it is determined to be visceral fat type.

At step S12 the result of calculation: total fat area of abdomen, subcutaneous fat area, visceral fat area, fat rate of trunk portion, fat rate of whole body, and type of fat are displayed on the display unit 10. Then, at step S13 it is determined whether the measurement switch 10 is depressed within fixed time period while the display unit 10 continues to display them. If the switch is depressed it is interpreted that the measurement is again performed. Then, the routine returns to step S3 to start the measurement. However, if the switch is not depressed within the fixed time period then the apparatus is automatically turned OFF.

It is preferable that the electrodes of the impedance measurement electrode system 9 are disposed on the front surface of abdomen to span as wider area as possible for widening the measurement region. In other words, preferably, the current supplying electrodes 7a and 7b are spaced apart as far as possible from each other on the front surface of abdomen so that they are disposed near both sides of abdomen. The same is true for the voltage measurement electrodes 8a and 8b. In this embodiment, however, the distance between the electrodes of the impedance measurement electrode system 9 is substantially fixed and the dimension of abdomen is reflected by the lateral width of abdomen. Accordingly, the distance between the current supplying electrodes 7a and 7b is preferably determined to suit to a person under test having small lateral width. In this embodiment this is determined to be 24 cm.

If the distance between the voltage measurement electrodes 8a and 8b would be shorter the measurement region, of course, becomes narrower even if the current supplying electrodes 7a and 7b are sufficiently spaced apart. Furthermore, as is well known, the distance between each current supplying electrode 7a, 7b and the adjacent voltage measurement electrodes 8a, 8b should generally be at least twice the subcutaneous fat thickness. Otherwise, the impedance measured may be affected only by subcutaneous fat tissue without inclusion of effect of muscle tissue.

Accordingly, in this embodiment, the distance between the current supplying electrode 7a and the voltage measurement electrode 8a, and between the current supplying electrode 7b and the voltage measurement electrode 8b is determined to be 8 cm in order to suit to a person under test having thicker subcutaneous fat of 4 cm.

### Second Embodiment

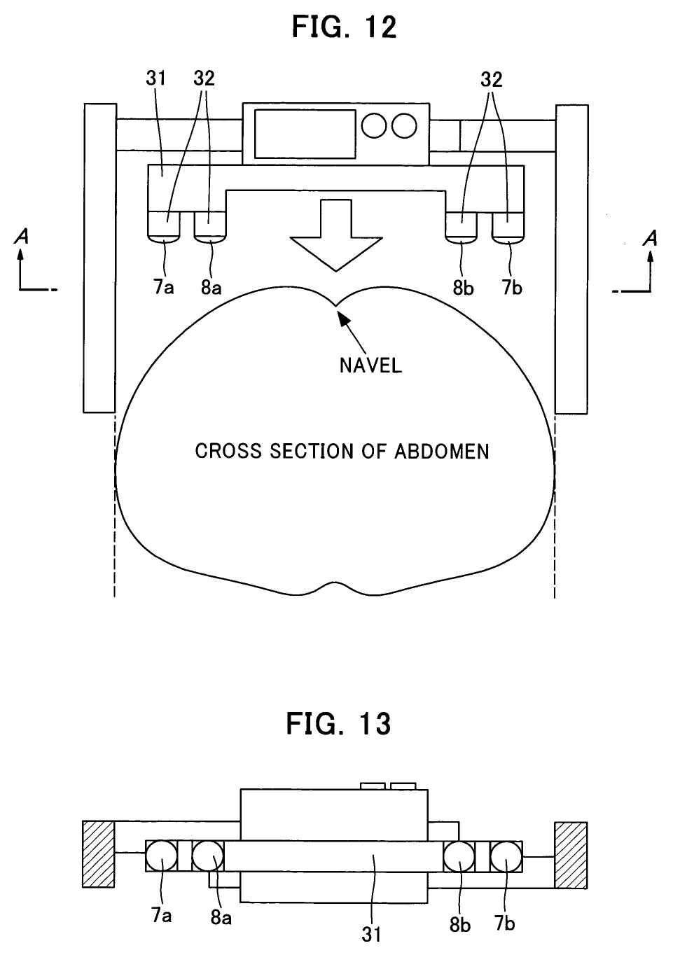

A second embodiment of the present invention is substantially same in construction and operation as the first embodiment as described above, except that the second embodiment includes different configuration of an electrode support unit and different disposition of an impedance measurement electrode system on the electrode support unit. Referring to FIGS. 12 and 13, instead of the flexible electrode support unit 3 of the first embodiment, a rigid electrode support unit 31 is provided according to the second embodiment in which a pair of current supplying electrodes 7a, 7b and a pair of voltage measurement electrodes 8a, 8b are mounted to an abdomen contact surface of the rigid electrode support unit 31 via flexible members 32 which allow said electrodes to freely be changed in contact direction to abdomen of a person under test. In particular, a recess is formed in the rigid electrode support unit 31 between the voltage measurement electrodes 8a and 8b, which recess accommodates a rounded center portion of front surface of abdomen of the person under test so that a surface of the rigid electrode support unit 31 on which the electrodes 7a, 7b, 8a, 8b are mounted is directed toward the front surface of abdomen of the person under test as the abdomen contact surface. As the result, the electrodes can closely be made contact to the front surface of abdomen of the person under test.

In case where the abdomen contact surface of the rigid electrode support unit 31 can surly be made contact to the front surface of abdomen of the person under test then the electrodes of the impedance measurement electrode system 9 may directly be mounted to the abdomen contact surface of the rigid electrode support unit 31 without any flexible members 32, which can lower the manufacturing cost of the apparatus.

### Third Embodiment

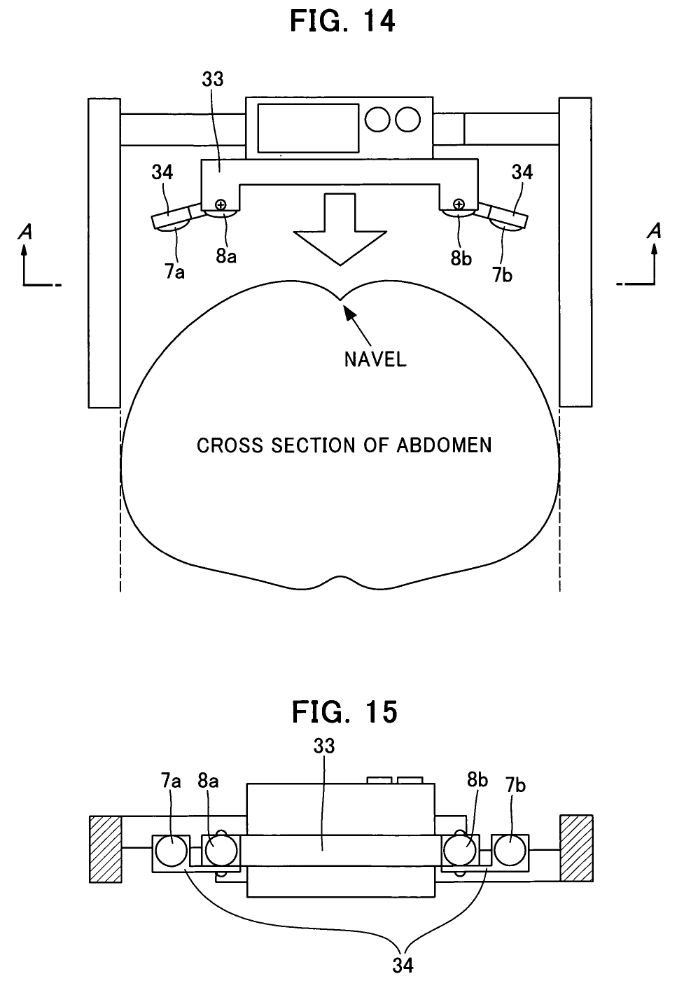

In the same manner as the second embodiment as above, a third embodiment of the present invention is substantially same in construction and operation as the first embodiment, except that the third embodiment includes different configuration of an electrode support unit and different disposition of an impedance measurement electrode system on the electrode support unit. Referring to FIGS. 14 and 15, the apparatus comprises an electrode support unit 33 on which voltage measurement electrodes 8a and 8b are fixedly mounted. A pivot member 34 is each provided for pivotally moving about each of the voltage measurement electrodes 8a and 8b and each of current supplying electrodes 7a and 7b is mounted to an end portion of the pivot member 34. The direction of the current supplying electrodes 7a and 7b is manually set to suit to front surface of abdomen of a person under test.

Inversely, the current supplying electrodes 7a and 7b may fixedly be mounted while the voltage measurement electrodes 8a and 8b may pivotally be moved.

### Fourth Embodiment

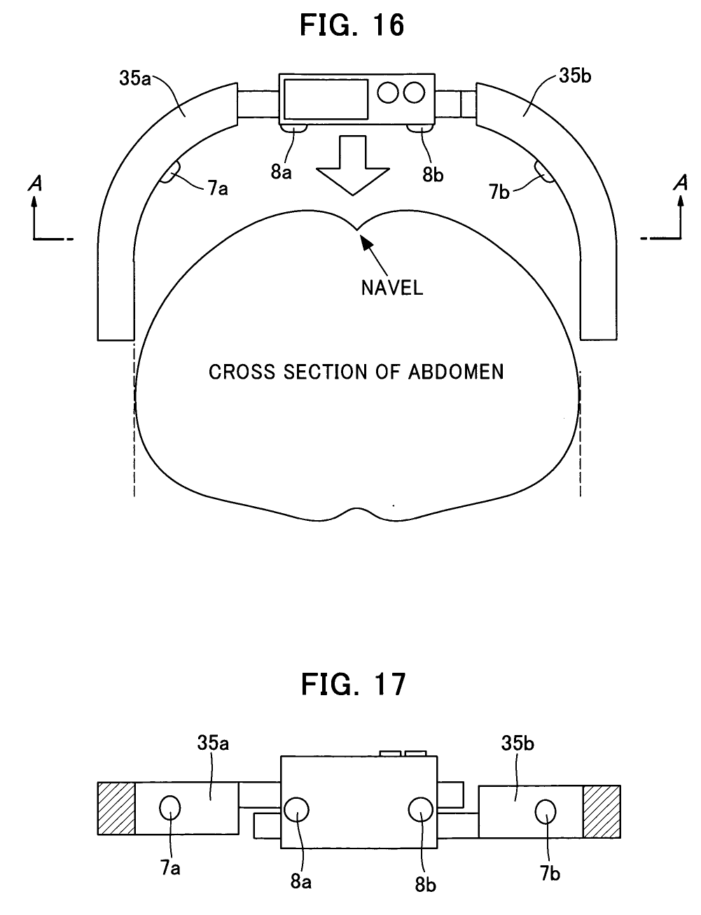

A fourth embodiment of the present invention is substantially same in construction and operation as the first embodiment as described above, except that the fourth embodiment includes different configuration of an electrode support unit and left and right arm portions as well as different disposition of an impedance measurement electrode system on the electrode support unit. Referring to FIGS. 16 and 17, electrode support arm portions 35a and 35b are formed by integrating the electrode support unit 3 with the left and right arm portions 4a, 4b in the first embodiment. Furthermore, some sliding mechanism is included in the body portion 2, in the same manner as the left and right arm portions 4a, 4b in the first embodiment. The electrode support arm portions 35a and 35b are curved to generally suit to the shape of front surface of abdomen of a person under test.

In order to dispose the impedance measurement electrode system 9 the current supplying electrodes 7a and 7b are mounted to the curved electrode support arm portions 35a and 35b at the positions corresponding to left and right sides of abdomen of the person under test. The voltage measurement electrodes 8a and 8b are mounted to the abdomen contact surface of the body portion 2 at the predetermined positions thereon. In this embodiment the distant between the voltage measurement electrodes 8a and 8b is 8 cm.

Upon measurement the electrode support arm portions 35a and 35b are sidably moved to suit to lateral width of abdomen of the person under test, and then, they are pressed against abdomen of the person under test, together with the body portion 2, so that the impedance measurement electrode system 9 is properly contact to abdomen of the person under test.

### Fifth Embodiment

In the first to fourth embodiment due to the fact that the impedance measurement region defined by the range within which the electrodes of the impedance measurement electrode system 9 can be contact is fixed in dimension relative to the lateral width of abdomen of the person under test then there may actually be variation in dimension of measurement region depending on the different persons under test. In order to overcome such fact, correction is made using lateral width of abdomen as the correction factor so that the measurement region can be considered kept constant irrespective of the different persons under test.

On the other hand, a fifth embodiment of the present invention provides a body fat measurement apparatus in which contact positions of electrodes of an impedance measurement electrode system 9 on abdomen of a person under test may be shifted according to the dimension of abdomen, which obviates measurement and entering of lateral width of abdomen.

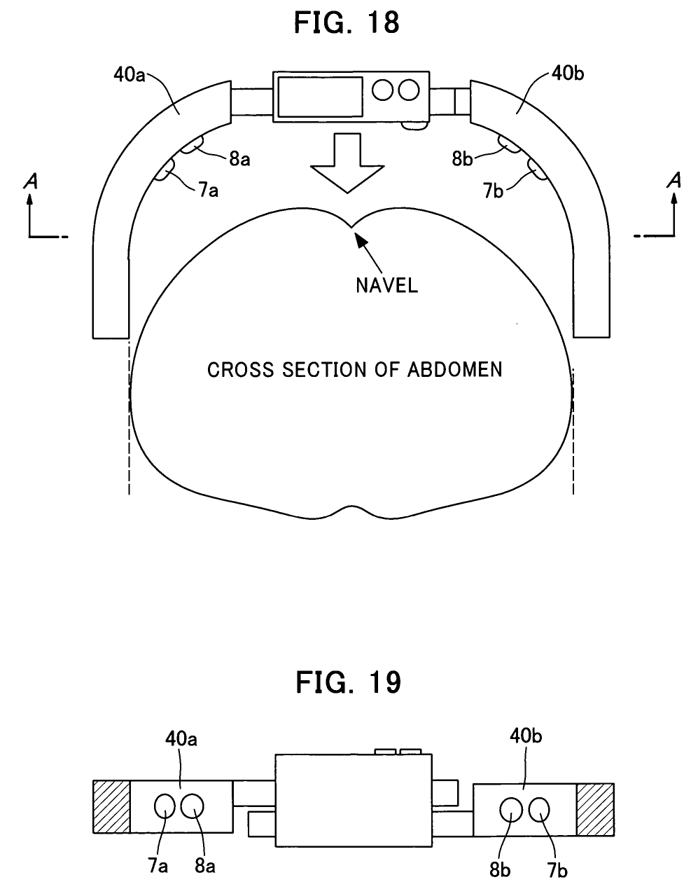

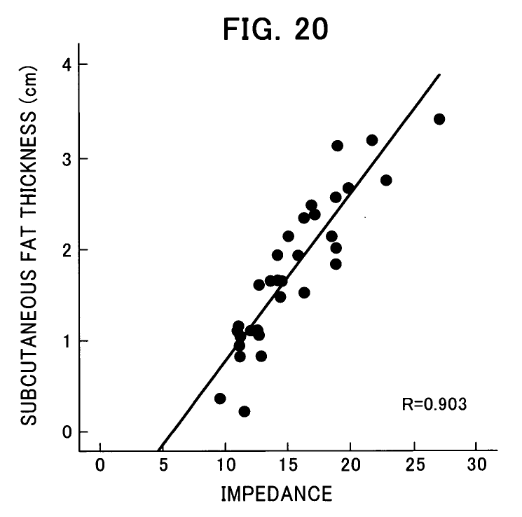

Referring now to FIGS. 18 and 19, the body fat measurement apparatus according to the fifth embodiment will be described. The elements having same construction and operation as those in the first to fourth embodiments are designated by the like reference numbers.

As shown in FIG. 18, electrode position correction arm portions 40a and 40b are curved to generally suit to the shape of front surface of abdomen of a person under test and are supported on a sliding mechanism in a body portion 2 so that they are sidably moved along the lateral width of abdomen, in the same manner as the electrode support arm portions 35a and 35b in the fourth embodiment as shown in FIG. 16. As described above, the electrode support arm portions 35a and 35b are configured so that the current supplying electrodes 7a and 7b are movable, but the voltage measurement electrodes 8a and 8b are fixed. Therefore, a correction according to lateral width of abdomen is necessary, which is conducted in such manner that the arm moving distance for which the arm portions have been moved is measured with an encoder 16 in the body portion 2, on the basis of which the lateral width of abdomen is automatically calculated and the correction factor is entered.

The electrode position correction arm portion 40a is provided with a current supplying electrode 7a and a voltage measurement electrode 8a. In the same manner, the electrode position correction arm portion 40b is provided with a current supplying electrode 7b and a voltage measurement electrode 8b. In this embodiment, the distance between the electrodes 7a and 8a and between the electrodes 7b and 8b is set at 8 cm.

As shown in FIG. 18, upon measurement the electrode position correction arm portions 40a and 40b are sidably moved to suit to lateral width of abdomen of the person under test and are pressed to the abdomen. As the result, the electrodes of the impedance measurement electrode system 9 can be contact with the abdomen of the person under test at such positions that suit to the dimension of abdomen. This obviates the need of entering the lateral width of abdomen as the correction factor.

The block diagram of the body fat measurement apparatus of this embodiment is same as that of the apparatus of the first embodiment as shown in FIG. 3, except that the encoder 16 is removed in this embodiment.

Furthermore, operation of the apparatus of the fifth embodiment is generally same as that of the first embodiment, as shown in the flow chart of FIG. 6, except that lateral width of abdomen is not used as the correction factor in the regression equations used for calculating the index of body fat at steps S5 to S9. The regression equation obtained as the result of correlation of the index of body fat calculated according to “CT,” or “DXA” method with “Z₅₀” and “Z_{6.25}” provided by impedance measurement is retrieved from ROM 14 in the same manner as the first embodiment.

In the first to fifth embodiment determination of whether the type of fat is subcutaneous or visceral at step S11 in flow chart of FIG. 6 is performed on the basis of difference between subcutaneous fat area and visceral fat area, as described above. Alternatively, in clinical treatment using “CT,” method, etc. the type of fat may be determined on the basis of ratio “V/S” where “V” is visceral fat area and “S” is subcutaneous fat area. In particular, the type of fat is determined to be visceral fat if “V/S”>0.5, and subcutaneous fat if “V/S”≦0.5.

Moreover, it may be possible that the body portion 2 is provided with some input unit for entering personal data of age, sex, etc. of a person under test and the regression equations for calculating the index of body fat are classified depending on age, sex, etc. and stored in ROM 14. As the result, more proper regression equation can be used to produce measurement result with higher precision.

The thickness of subcutaneous fat can also be calculated. In general, it is well known that there is higher correlation between the thickness of subcutaneous fat calculated according to “CT” method and the impedance of abdomen measured using the current at frequency of 50 kHz. As described above regarding the principle of measurement of abdomen's impedance with reference to FIGS. 5 to 7, however, when using lower frequency current the impedance value more strongly affected by subcutaneous fat tissue is resulted than at the time when using higher frequency current. Therefore, the regression equation obtained as the result of correlation of the impedance at lower frequency with the thickness of subcutaneous fat calculated by “CT” method can be used to calculate the thickness of subcutaneous fat with higher precision.

FIG. 22 is a graph illustrating correlation between the subcutaneous fat thickness and the impedance measured with lower frequency current. There is higher correlation resulted with correlation factor of 0.903.

As described above, the current at two frequencies: lower frequency of 6.25 kHz and higher frequency of 50 kHz is supplied to the current supplying electrodes 7a and 7b. If the number of frequencies is increased it is possible to more precisely reflect relationship between muscle tissue and subcutaneous fat tissue, which can increase precision with which the measurement is conducted. Even with one frequency used the impedance affected by both muscle tissue and subcutaneous fat tissue can be obtained, which provides facilitated measurement.

1. A body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated, said apparatus further comprising:
an electrode support unit;
a lateral width measurement unit; and
an arithmetic unit, wherein
said electrode support unit supports the impedance measurement electrode system so that the electrodes thereof are contact with front surface of the abdomen of the person under test for measuring impedance of the abdomen,
said lateral width measurement unit measures lateral width of the abdomen of the person under test, and
said arithmetic unit calculates the index of body fat of the person under test on the basis of the impedance of the abdomen and the lateral width.

2. A body fat measurement apparatus according to claim 1 in which said electrode support unit is formed from flexible member.

3. A body fat measurement apparatus comprising an impedance measurement electrode system including a pair of current supplying electrodes and a pair of voltage measurement electrodes, those electrodes adapted for contact with peripheral surface of abdomen of a person under test to measure impedance of the abdomen, on the basis of which an index of body fat of the person under test is calculated, said apparatus further comprising:
an electrode support unit;
an electrode position shift unit; and
an arithmetic unit, wherein
said electrode support unit supports the impedance measurement electrode system so that the electrodes thereof are contact with front surface of the abdomen of the person under test for measuring impedance of the abdomen,
said electrode position shift unit moves the electrode support unit according to lateral width of the abdomen so that the electrodes of the impedance measurement electrode system are set at suitable position on the front surface of the abdomen, and
said arithmetic unit calculates the index of body fat of the person under test on the basis of the impedance of the abdomen.

4. A body fat measurement apparatus according to any one of claims 1 to 3 in which said electrode support unit supports the impedance measurement electrode system so that the pair of voltage measurement electrodes is positioned between the pair of current supplying electrodes.

5. A body fat measurement apparatus according to any one of claims 1 to 3 in which said electrode support unit includes an electrode direction change unit, said electrode direction change unit allows at least one of the pair of current supplying electrodes and the pair of voltage measurement electrodes to change in contact direction in order to suit to the shape of the abdomen.

6. A body fat measurement apparatus according to any one of claims 1 to 3 in which it further comprises a frequency switching unit, said frequency switching unit switches frequency of AC current supplied to the pair of current supplying electrodes in order to conduct measurement of impedance at two or more frequencies of at least one of higher and lower frequency ranges.

7. A body fat measurement apparatus according to any one of claims 1 to 3 in which said index of body fat includes at least one of the followings: total fat area of abdomen, subcutaneous fat area, subcutaneous fat thickness, visceral fat area, fat rate of trunk portion, fat rate of whole body, and ratio of subcutaneous fat to visceral fat.
